(19) United States
(12) Patent Application Publication
(10) Pub. No.: US 2003/0152955 A1
(43) Pub. Date: Aug. 14, 2003

(54) Method for identifying transposons from a nucleic acid database

(76) Inventor: Thomas Bureau, Quebec (CA)

Correspondence Address: OGILVY RENAULT, 1981 MCGILL COLLEGE AVENUE, SUITE 1600, MONTREAL, QC, H3A2Y3, CA

(21) Appl. No.: 10/203,640

(22) PCT Filed: Feb. 26, 2001
(86) PCT No.: PCT/CA01/00251

(30) Foreign Application Priority Data
Feb. 24, 2000 (US) 60184650

(51) Int. Cl. C12Q 1/68; G06F 19/00; G01N 33/48; G01N 33/50; C12N 15/74

(52) U.S. Cl. 435/6; 702/20; 435/473

(57) ABSTRACT

The invention relates to a method for determining if repetitive sequences from nucleic acid sequence databases are bona fide transposons.

### BACKGROUND OF THE INVENTION

(a) Field of the Invention

The invention relates to a method for determining if repetitive sequences from nucleic acid sequence databases are bona fide transposons.

(b) Description of Prior Art

Transposons are fundamental components of most eukaryotic genomes contributing to their size, structure, and variation. They can be classified into two general classes distinguished primarily by their structural features and mechanism of mobility.

Class I elements are generally referred to as retroelements and move via the reverse transcription of an RNA intermediate. Retroelements include such diverse elements as retroviruses, retrotransposons (e.g., gypsy and copia of Drosophila), Long and Short Interspersed Nuclear Elements (LINEs and SINEs, respectively), and processed pseudogenes. The copy number of retroelements can be very high representing the majority of large eukaryotic genomes.

Class II elements are commonly referred to as inverted-repeat transposons as they have a usually short terminal inverted repeats. They move by a so-called “cut-and-paste” mechanism that does not involve an RNA intermediate nor reverse transcriptase. Instead the excision (cut) and reinsertion (paste) is mediated by an element-encoded transposase. Plant transposons can be classified into eight superfamilies: the class I elements—SINEs, LINEs, copia-like retrotransposons, and gypsy-like retrotransposons; the class II elements Ac-like, CACTA-like, Mutator (including MUtator-like Elements or MULEs) and MITEs (Miniature Inverted-repeat Transposable Elements).

Transposons have often been viewed as “junk” DNA presumably since they serve no function to their hosts. However, a handful of studies challenge this paradigm and suggest that transposons may have an important evolutionary role in generating variation. For example, an enhancer sequence contained within a cryptic retrotransposon insertion in the 5′ flanking region of the murine sip gene confers androgen-specific regulation. Likewise, a retroelement insertion in the 5′ flanking region of the human Amy1 gene confers salivary gland-specific expression. In addition, an endogenous retroviral LTR induces steroid-mediated alternative splicing of the human leptin receptor OBR mRNA. The protein encoded by the alternatively-spliced transcripts lacks a domain required for intracellular signal transduction suggesting a regulatory involvement. Although the functional significance is not known, some transposons contribute to the coding capacity of some wild-type genes. In general, however, the actual role of transposons in the evolution of gene structure, expression, and regulation still awaits elucidation.

The development of the RFLP (Restriction Fragment Length Polymorphism) technique as a molecular mapping tool has facilitated the rapid evolution of genome mapping and fingerprinting technologies. This evolution has resulted in the development of such cornerstone techniques as RAPD (Randomly Amplified DNA Polymorphism) and AFLP (Amplified Fragment Length Polymorphism). Modern genome mapping and fingerprinting techniques have been made even more powerful by exploiting the use of repetitive genomic anchor sequences usually derived from retroelements (Flavell et al., Plant journal 16:643-649, 1998; and Zietkiewicz E., et al., Proceedings of the National Academy of Sciences (USA) 89: 8448-8451, 1992), short sequence repeats (SSRs), and MITEs. Clearly, these techniques are limited only by the identification of the genomic interspersed repetitive sequences, namely transposon sequences, used to design primers for PCR-based mapping technologies.

For the most part, the vast majority of transposons are inactive (e.g. transcriptionally silent and/or not mobile) during the development of their hosts. This may be a result of purifying selection against element activity since transposon insertions may lead to deleterious mutations or, more generally, lowered fitness of the host. However, many transposons can be activated when subjected to various types of environmental stresses (Wessler, Current Biology 6:959-961, 1996; Hirochika, Plant Molecular Biology 35: 231-240, 1997). In fact, genetic analyses of maize unstable mutant phenotypes by the activation of the Ac transposon by UV and gamma irradiation were conducted. Later, the maize Ac and Spm elements were also found to be activated in cell culture. More recently, protoplast formation and cell culture was determined to activate plant copia-like retrotransposons (e.g. Tnt in tobacco and Tto in rice). Agrobacterium-mediated transformation was shown to activate the Ac-like element Tag1 in Arabidopsis. Intriguingly, element activation during Agrobacterium-mediated transformation, protoplast formation and/or cell culture has been suggested to underlie the generation of some clonal variants in regenerated, including transgenic, plants. Moreover, biotic and abiotic stresses have also been observed to activate a wide range of transposons from other eukaryotes.

Stressed-induced activation of transposons has important evolutionary implications. As a major source of spontaneous mutations, transposons have been implicated as a source in the generation of naturally occurring genetic variation. In fact, there are a growing number of reports documenting transposons contributing cis-factors and structural components to wild-type genes. In addition, induction of retroelement activity in response to viral infection is proposed to be a mechanism by which horizontal transmission can occur.

Activation of endogenous transposons has implications in the development of functional genomics technologies. Transposon-mediated mutagenesis is the tool of choice for plant gene “knockouts” and the basis of several gene isolation approaches. The latter may involve the introduction of engineered transposons. The utility of such an approach is obviously limited by available transformation protocols and the robustness of element activity in the host. Recently, activation of endogenous elements has proven to be very effective in both gene isolation and characterization. This approach is only limited by the identification of “active” endogenous transposons.

Many transposons have been identified as the causal agents underlying mutations by means of traditional molecular genetics approaches.

In the actual state of the art, transposons identification can not be done without experimentation in laboratory to test if a repetitive sequence and/or structure related to transposons is acting as facilitating gene transport. Such experimentation is very costly and time consuming.

It would be highly desirable to be provided with method for mining transposons from nucleic acid and protein databases.

### SUMMARY OF THE INVENTION

One aim of the present invention is to provide a method for determining if a nucleic acid sequence is a transposon, the method comprising the steps of:

a) identifying a location in a nucleic acid database at which a potential transposon to be identified may be found;

b) selecting at least one flanking region sequence of the potential transposon;

c) searching the database for at least one match of the flanking region sequence selected;

d) comparing a target site nucleic acid sequence and both a leading and a trailing flanking region sequence between the potential transposon and the match.

e) determining the value as a function of the comparison.

In accordance with a preferred embodiment of the present invention, there is provided the method of the present invention, wherein step a) is completed by querying a nucleic acid database to find repetitive sequences, queries including genomic sequences being selected from the group consisting of non-coding regions, regions annotated with low similarity to genes or with predicted exons, sequences annotated as previously identified transposon, sequences annotated as having an open reading frame as part of a previously identified transposon, sequences annotated as having a putative transposon and sequences annotated as, having a repetitive region, the queries being executed with one or more, search algorithms and the queries retrieving regions with significant sequence similarity.

In accordance with a preferred embodiment of the present invention, there is provided the method of the present invention, wherein the search algorithm is Basic Local Alignment Search Tool (BLAST).

In accordance with a preferred embodiment of the present invention, there is provided the method of the present invention, wherein step a) is also completed by screening sequences for structures indicative of transposons, the structures including terminal inverted repeats (TIRs), long terminal direct repeats (LTRs), genes related to mobility and target site duplications (TSDs), the screening using one or more structure identifier algorithms facilitating structural analysis.

In accordance with a preferred embodiment of the present invention, there is provided the method of the present invention, wherein the structure identifier algorithms are GAP, REPEAT and STEMLOOP.

In accordance with a preferred embodiment of the present invention, there is provided the method as claimed in any one of claims 1-5, wherein the value indicative of a nucleic acid sequence being a transposon is based on correspondence of insertion sequence to a gap in pairwise alignment coupled to the presence of a target site duplication, said correspondence being determined using significant sequence similarity criteria.

One other aim of the present invention is to provide a computer program product comprising code means adapted to perform all steps of the method of the present invention, embodied on a computer readable medium or embodied as an electrical or electro-magnetic signal.

A further aim of the present invention is to provide a computer data signal embodied in a carrier wave and representing sequences of instructions which, when executed by a processor cause the processor to perform the method of the present invention.

Another aim of the present invention is to provide an apparatus for determining a value indicative of a nucleic acid sequence being a transposon comprising:

means for identifying a location in a nucleic acid database at which a potential transposon to be identified may be found;

means for selecting at least one flanking region sequence of the potential transposon;

means for searching said database for at least one match of the at least one flanking region sequence;

means for comparing a target site nucleic acid sequence and both leading and trailing ones to the flanking region sequences between the potential transposon and at least one match;

means for determining the value as a function of the comparising.

In accordance with another embodiment of the present invention, there is provided the apparatus of the present invention, wherein identifying a location in a nucleic acid database is completed by querying a nucleic acid database to find repetitive sequences, queries including genomic sequences being selected from the group consisting of non-coding regions, regions annotated with low similarity to genes or with predicted exons, sequences annotated as previously identified transposon, sequences annotated as having an open reading frame as part of a previously identified transposon, sequences annotated as having a putative transposon and a sequence annotated as having a repetitive region, the queries being executed using one or more search algorithms and the queries retrieving regions with significant sequence similarity.

In accordance with another embodiment of the present invention, there is provided the apparatus of the present invention, wherein the search algorithm is BLAST.

In accordance with another embodiment of the present invention, there is provided the apparatus of the present invention, wherein identifying a location in a nucleic acid database is also completed by screening sequences for structures indicatives of transposon, the structures including TIRs, LTRs, genes related to mobility and TSDs, the screening using a structure identifier algorithm facilitating structural analysis.

In accordance with another embodiment of the present invention, there is provided the apparatus of the present invention, wherein the structure identifier algorithms are GAP, REPEAT and STEMLOOP.

In accordance with another embodiment of the present invention, there is provided the apparatus of the present invention, wherein the value indicative of a nucleic acid sequence being a transposon is based on correspondence of the insertion sequence to a gap in pairwise alignment and the presence of a target site duplication, said correspondence being determined using significant sequence similarity criteria.

Mined transposons can be used to genotype a nucleic acid sequence using polymerase chain reaction (PCR) amplification or hybridization based protocols and sequences unique to the mined transposons. In accordance with the present invention, the mined transposon can be used in fingerprinting or linkage studies. Active mined transposons can also be used for the isolation of novel genes, for the production of mutated or “knockout” genes, and the delivery of engineered genes. With the present invention, protocols based on mined transposons will be fundamentally important in genomics and biotechnical approaches.

For the purpose of the present invention the following terms are defined below.

The term “transposon” is intended to mean a type of genetic element that is capable of movement. Movement may be through a DNA or RNA intermediate. Transposons are also referred to as mobile genetic elements, transposable elements, mobile elements, and jumping genes. Most transposons produce a target site duplication (TSD) upon insertion.

The term “Ac-like transposon” is intended to mean a superfamily of transposons with features similar to the maize Activator transposon and other previously reported Activator-like transposons. Ac-like elements are usually less than 10 kilobases in length, have a short perfect or degenerate terminal inverted repeat, and have an eight base pair target site preference. Some Ac-like elements harbor open reading frame(s) with similarity to the maize Activator transposase.

The term “CACTA-like” is intended to mean a superfamily of transposons with features similar to the maize En/Spm transposon and other previously reported En/Spm-like elements. CACTA-like elements are usually less than 20 kilobases in length, have a short perfect or degenerate terminal inverted repeat, and have a three base pair target site preference. Some CACTA-like elements harbor open reading frame(s) with similarity to the maize En/Spm transposase(s).

The term “MULE” is intended to mean a superfamily of transposons found in many eukaryotic organisms including Arabidopsis. MULEs are usually less than 20 kilobases in length, have no target sequence preference, have a target site size preference of 9-12 base pairs. Many, but not all, MULEs harbor genes that code for putative Mutator-like transposase.

The term “SINE” is intended to mean short interspersed nuclear element. These elements are structurally similar to structural cellular RNA genes. SINES are usually terminated by an “A”-rich, “AT”-rich. or simple sequence repeat (SSR) sequence, have a target site sequence of less than 50 base pairs. Some SINEs harbor sequences with similarity to the A and B promoters of structural RNA genes. Some SINEs have a tripartite structure, that is i) a component with similarity to a structural RNA gene, ii) a component that has no sequence similarity to a structural RNA gene, and iii) a component that consists of an “A”-rich, “AT”-rich, or SSR sequence.

The term “LINE” is intended to mean long interspersed nuclear element. These elements are usually less than 20 kilobases in length, have many of the coding domains found in copia-like, gypsy-like, and retroviral-like retrotransposons, are usually terminated by an “A”-rich, “AT”-rich, or SSR sequences, and is flanked by a direct repeat of less than 50 base pairs. Unlike copia-like, gypsy-like, and retroviral-like retrotransposons, LINEs do not have long direct repeats at their termini.

The term “copia-like retrotransposons” is intended to mean any transposon with nucleic acid or amino acid sequence similarity to the copia transposon of Drosophila or the Ty1 transposon of yeast, copia-like retrotransposons are usually less than 20 kilobases in length, have long terminal repeats (LTRs) from 50 base pairs to 5 kilobases in length, and have a target site sequence preference of five base pairs.

The term “gypsy-like retrotransposons” is intended to mean any transposon with nucleic acid or amino acid sequence similarity to the gypsy transposon of Drosophila or the Ty3 transposon of yeast, gypsy-like retrotransposons are usually less than 20 kilobases in length, have long terminal repeats (LTRs) from 50 base pairs to 5 kilobases in length, and have a target site sequence preference of five base pairs.

The term “Basho” is intended to mean a superfamily of transposons mined from Arabidopsis genome sequence and from maize genomic gene sequence. These elements are less than 5 kilobases in length, have at least a two base pair terminal inverted repeat (e.g. 5′-CA . . . GT-3′), a target site preference for the mononucleotide “T” and are moderately to highly abundant in the genome. The previously described repetitive sequences referred to as Aie (Arabidopsis insertion sequence) and AthE1 (Arabidopsis element 1) have nucleic acid sequence similarity to some members of the Basho superfamily of transposons.

The term “VIRMIN transposon” is intended to mean VIRtually MINed transposon. VIRMIN transposons were identified by computer-assisted sequence similarity searches and computer-assisted sequence analysis and include members of the Ac-like, En/Spm-like, MULE, MITE, SINE, LINE, copia-like retrotransposons, gypsy-like retrotransposons, and Basho superfamily of transposons. VIRMIN transposons also refer to newly identified transposons that do not fit any of the previously known superfamily of transposons.

The term “RESite” is intended to mean sequences that are Related to Empty Site. There are four steps for determining RESite. First, sequences immediately flanking the putative insertion sequence are used as queries in database searches. Queries can either be the 5′ flanking region, 3′ flanking region or a query that contains both the 5′ and 3′ flanking regions with the putative insertion sequence edited out. Second, genomic regions sharing high similarity with the query are subjected to a pairwise comparison. The searches may identify sequences with high similarity form paralogous, orthologous sequences or regions within repetitive sequences. Third, a gap corresponding to the absence of the putative insertion sequence is used as starting point to delimit the termini. The algorithms used in pairwise alignments should only be used as guides to begin making the final alignment. Often these algorithms are constrained by the size of the gaps and level of sequence similarity. Manual alignment, base-by-base, is almost always required. Fourth, the sequences immediately flanking the localized insertion sequence are examined for direct repeats. Almost all transposons create a target site duplication immediately flanking the element upon insertion. The target site can be one base pair to over 20 base pairs in length depending on the transposon type. Together the correspondence of the insertion sequence to a gap in the pairwise alignment and the presence of a target site duplication provides convincing evidence that the putative insertion sequence is a bona fide transposon.

The term “eukaryote” or “eukaryotic organism” is intended to refer to plants, animals, and fungi.

The measure of significant sequence similary used in the present application is a BLAST score of >80.

BLAST is intended to mean Basic Local Alignment Search Tool and it is a standard sequence similarity algorithm available through the National Center of Biological Information (NCBI: http://www.ncbi.nlm.nih.gov/blast/).

The term “Basepair” is intended to mean any possible pairing between bases in opposing strands of DNA or RNA. Adenine pairs with thymine in DNA, or with uracil in RNA; and guanine pairs with cytosine.

The term “Exons” is intended to mean the protein-coding DNA sequences of a gene.

The term “Introns” is intended to mean the sequence of DNA bases that interrupts the protein-coding sequence of a gene; these sequences are transcribed into RNA but are edited out of the message before it is translated into protein.

The term “Open reading frame (ORF)” is intended to mean a series of DNA codons, including a 5′ initiation codon and a termination codon, that encodes a putative or known gene.

The term “Polymerase chain reaction (PCR)” is intended to mean a method for amplifying a DNA base sequence using a heat-stable polymerase and two primers, one complementary to the (+)-strand at one end of the sequence to be amplified and the other complementary to the (−)-strand at the other end. The faithfulness of reproduction of the sequence is related to the fidelity of the polymerase.

The term “Expressed Sequence Tag (EST)” is intended to mean a partial sequence of a clone, randomly selected from a cDNA library and used to identify genes expressed in a particular tissue.

The term “Sequence Tagged Site (STS) is intended to mean a short (200 to 500 basepairs) DNA sequence that has a single occurrence in the human genome and whose location and base sequence are known.

The term “Paralogous” is intended to mean homologous proteins that perform different but related functions within one organism.

The term “Orthologous” is intended to mean homologous proteins that perform the same function in different species.

The term “Target site nucleic acid sequence” is intended to mean a nucleic acid sequence which is duplicated by the insertion of a transposon.

The term “Target site duplicate” is intended to mean the duplicate of the Target site nucleic acid sequence as defined above.

The term “match” is intended to mean one hit from a database query where the nucleic acid sequences compared are of significant similarity.

The term “flanking region” is intended to mean the 5′ flanking region, the 3′ flanking region or both the 5′ and 3′ flanking region. It can also be intended to mean a sequence region distant of a few basepairs of the 5′ and/or the 3′ in case where the putative transposon is not well known in order to avoid having a flanking region comprising part of the putative transposon.

“GAP” is a Pairwise comparison program that uses the algorithm of Needleman and Wunch (1970) to find the optimal global alignment of two sequences.

“REPEAT” is a repetitive sequence identification program that finds repeats within a sequence.

“STEMLOOP” is an RNA Secondary Structure program that finds stems, or inverted repeats, within a sequence. The user specifies the minimum stem length, minimum and maximum loop sizes, and the minimum number of bonds per stem.

### BRIEF DESCRIPTION OF THE DRAWINGS

FIG. 1A illustrates examples of RESites corresponding to mined elements for different groups of mined elements;

FIG. 1B illustrates RESites found for Basho insertions;

FIG. 2A illustrates similarities in structure between TIRs and TSDs (underlined) of an Arabidopsis MLE I member and Tc1/Mariner-like elements Pogo (Drosophila, gi 8354) and Tigger (human, gi 2226003); and

FIG. 2B illustrates an alignment of putative transposase for the Arabidopsis MLE I (gi 4262216) with transposases from Drosophila melanogaster PogoR11 (gi 2133672) and from human Tigger1 (gi 2226004).

FIG. 3 illustrates a pairwise alignment corresponding to mined transposon.

### DETAILED DESCRIPTION OF THE INVENTION

The present invention provides a method for mining and identifying transposon sequences from nucleic acid sequence databases. The usefulness of this method was determined by the mining of over 600 transposons from Arabidopsis thaliana genomic sequences. The vast majority of transposons were MITEs and members of a newly discovered superfamily of transposons referred to as Basho. These VIRtually MINed (VIRMIN) transposons can be used in many downstream applied technologies.

With the development of computer-based technologies, the vast majority of transposons are now “mined’ from DNA sequence databases. More efficient and automated DNA sequencing technologies and the efforts of numerous genome sequencing projects fuel the rapid growth of these databases. Many elements have been mined within intergenic regions in Arabidopsis, rice and maize. However, numerous elements have been found in very close proximity to plant genes. Of these elements, MITEs predominate.

### Advantages and Improvements over Existing Technology

The present invention offers an accurate, efficient, high throughput approach to identification of transposons compared to the use of standard genetic and molecular biological approaches. The transposon sequences discovered in the present invention greatly outnumber all of the plant transposon sequences previously reported. The transposons mined and characterized were found because of their close association with plant genes. Thus, these elements are unlikely to be confined to repetitive regions of genomes. The pervasiveness of VIRMIN transposons in the present application is of enormous value.

### Technical Descriptions

i) Computer-Based Mining of Transposons

Queries in database searches consisted of non-coding regions from genomic sequences, namely intergenic regions, introns, and untranslated regions. In addition, regions annotated with low similarity to genes or with predicted exons were included as queries. Some genomic sequences were annotated as having a) a previously identified transposon (as described in the scientific literature), b) an open reading frame as part of a previously identified transposon (i.e. transposase or reverse transcriptase), c) a putative transposon, or d) a repetitive region. These regions were also used as queries. The BLAST search algorithm was used as the primary mechanism to mine repetitive sequences. However, the FASTA search algorithm was also used with nucleic acid sequence queries. In addition, the search algorithm TFASTA was used with virtually translated nucleic acid sequences. BLAST (version 2.0) was accessed remotely at the National Center for Biotechnology Information (NCBI, http://www.ncbi.nlm.nih.gov/entrez/nucleotide.html) or locally at McGill University. All other algorithms for computer-assisted database searches and sequence analysis were accessed as part of the University of Wisconsin Genetics Computing Group (UWGCG) program suite at McGill University.

Based on the sequencing information available at the Arabidopsis Genome Initiative (AGI, http://genome-www.stanford.edu/Arabidopsis), a sample of annotated BAC, P1 or TAC clone sequences was selected for transposon mining. Sequence for these clones were accessed via the National Center for Biotechnology Information (NCBI, http://www.ncbi.nlm.nih.gov/entrez/nucleotide.html). A total of 243 annotated BAC clones (representing approximately 17.2 Mb) from each of the five chromosomes were retrieved for analysis. From these selected clones, sequences located between open reading frames (ORFs) annotated as genes and intron sequences larger than 500 base pairs were used as primary queries in BLAST searches. Regions with significant sequence similarity (BLAST scores>80) to at least 10 other Arabidopsis sequences and/or similarity to known transposable elements were noted for further investigation. Annotated similarity to transposons or features of transposons was also noted for investigation.

Sequences sharing significant similarity (BLAST scores>80) were compiled and screened for structures indicative of transposons. These include terminal inverted repeats, long terminal direct repeats, and flanking direct repeats (i.e. TSD). The algorithms GAP, REPEAT, and STEMLOOP facilitated structural analysis. Often with sequences sharing high sequence similarity the termini can be precisely mapped.

A novel technique named Related to Empty Site (RESite) was used to determine the actual termini of putative transposons and to document past mobile history. The RESite technique has four key steps. First, sequences immediately flanking the putative insertion sequence are used as queries in database searches. Queries can either be the 5′ flanking region, 3′ flanking region or a query that contains both the 5′ and 3′ flanking regions with the putative insertion sequence edited out. Second, genomic regions sharing significant sequence similarity with the query are subjected to a pairwise comparison. The searches may identify sequences with significant sequence similarity form paralogous, orthologous sequences or regions within repetitive sequences. Third, a gap corresponding to the absence of the putative insertion sequence is used as starting point to delimit the termini. The algorithms used in pairwise alignments should only be used as guides to begin making the final alignment. Often these algorithms are constrained by the size of the gaps and level of sequence similarity. Manual alignment, base-by-base, is almost always required. Fourth, the sequences immediately flanking the localized insertion sequence are examined for direct repeats. Almost all transposons create a target site duplication immediately flanking the element upon insertion. The target site can be one base pairs to over 20 base pairs in length depending on the transposon type. Together the correspondence of the insertion sequence to a gap in the pairwise alignment and the presence of a target site duplication provides convincing evidence that the putative insertion sequence is a bona fide transposon.

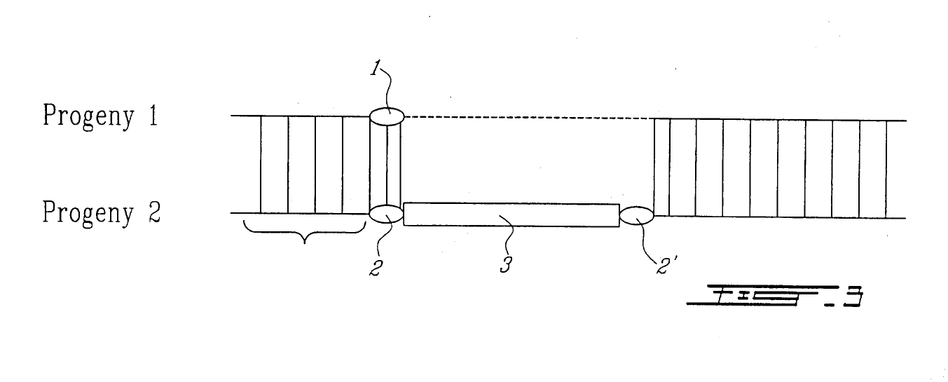

FIG. 3 illustrates pairwise alignments used in RESite technique to provide evidence that the putative insertion sequence is a transposon. In FIG. 3, (1) represents the target site nucleic acid sequence the “match” sequence, (2) represents the target site nucleic acid sequence of the sequence comprising the putative transposon, (2′) represents the target site duplicate at the end of the putative transposon, (3) represents the putative transposon and the bracket represents a possible flanking region as previously defined in the specification.

Whenever there were insufficient genomic sequences in the nucleic acid databases to implement RESite, a PCR-based approach was used to generate a genomic sequence. Basically, primers were designed from the 5′ and 3′ flanking regions of the putative transposon. The region between and including the 5′ primer and the 3′ primer is referred to as the Reference DNA Sequence (RDS). The region between and including the 5′ primer and the 3′ primer without the putative transposon sequence is referred to as Virtually-edited DNA Sequence (VDS). DNA fragments were amplified using these primers from genomic DNA of the organism containing the putative transposon and of closely related organisms to the organism containing the putative transposon. DNA fragments corresponding to the predicted size of the VDS were isolated, cloned and sequenced. If the sequenced DNA fragment shares sequence similarity to the RDS, then it was used in the RESite procedure.

In this way, repetitive nucleic acid sequences mined from nucleic acid databases were classified as transposons if they meet at least one of the following criteria: i) the mined repetitive nucleic acid sequence shares significant sequence similarity to a previously reported transposon, ii) the mined repetitive nucleic acid sequences has a structure similar to class I or II transposons as defined above, and/or iii) have defined termini and are flanked by direct repeats as determine by sequence analysis or by RESite.

ii) Plant Materials

Seeds for Arabidopsis thaliana ecotypes No-0, Sn-1, Ws Nd-1, Tsu-1, RLD1, Di-G, S96, Tol-0, Be-0 and Ler were obtained from Arabidopsis Biological Resource Center (HTTP://aims.cps.msu.edu/aims) and grown to maturity in a Sanyo growth cabinet at 20° C.

iii) Genomic DNA Isolation

Genomic DNA was extracted using a standard protocol.

iv) PCR Amplification for RESite

Oligonucleotides corresponding to the flanking sequences of the element were designed using the prime program from the UWGCG program suite. PCR amplifications were performed following standard procedures using AmpliTaq™ DNA polymerase (Perkin Elmer).

v) Cloning and Sequencing

PCR products were either gel purified or directly cloned into a modified pUC118 vector digested with Xcm1 (New England Biolabs). Ligations were carried out with T4 DNA ligase (GibcoBRL, Life Technologies) under the conditions suggested by the manufacturer. The cloned PCR products were subsequently sequenced using the standard procedures provided with SequiTherm EXCEL II DNA sequencing kit (Epicentre Technologies) with M13 forward and reverse primers.

### Results

a) Transposon Mining

17.2 megabases of Arabidopsis sequences were retrieved from 243 annotated BAC and P1 clones with representation on all 5 linkage groups. Regions less than 500 base pairs in length, ESTs and STSs were not included in our survey.

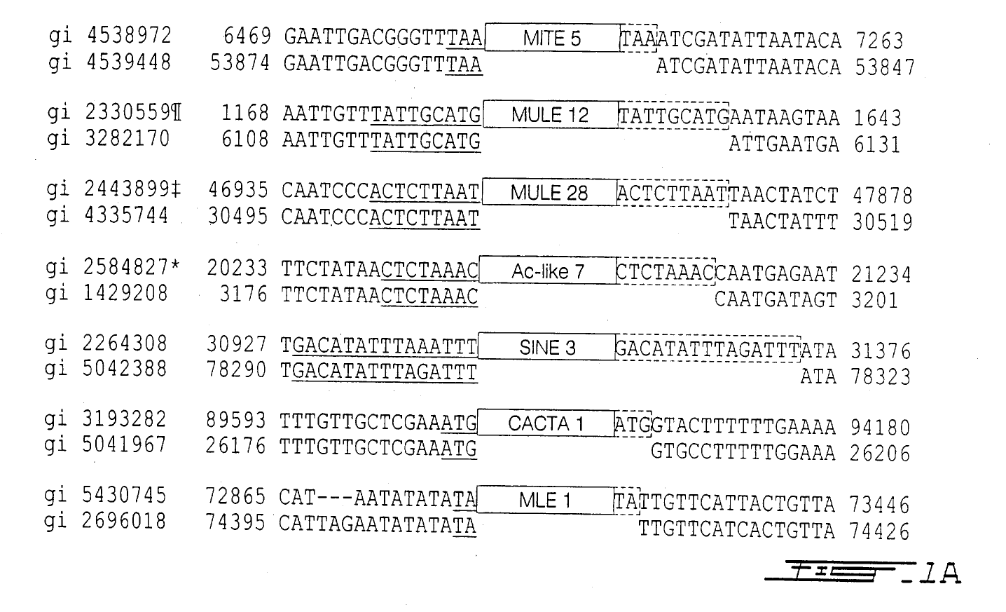

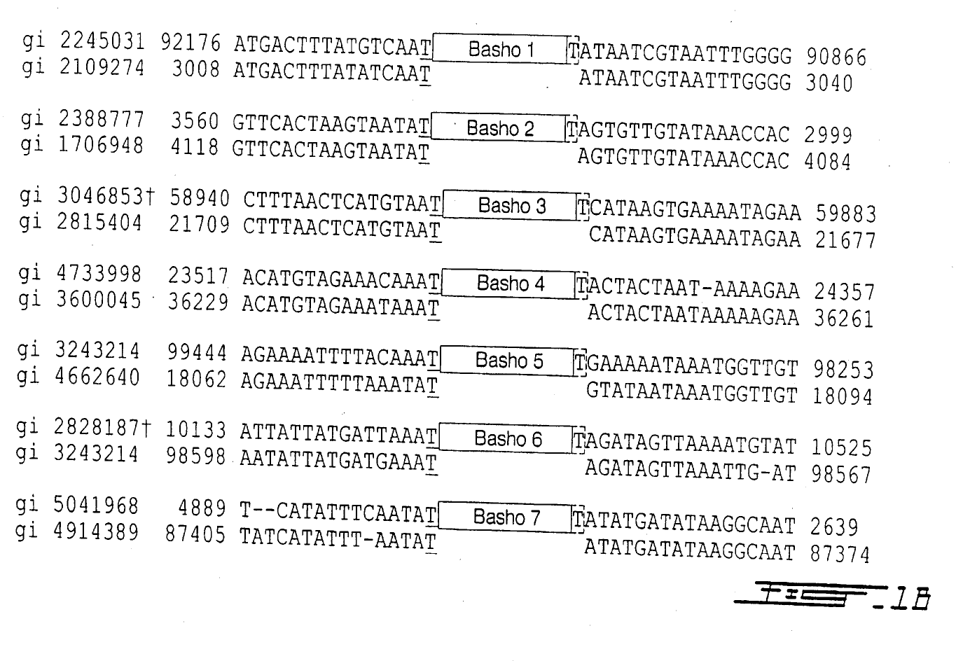

A total of 630 VIRMIN transposons were mined falling into eight basic groups (Table 1). The groups could be further divided into subgroups based on sequence similarity between group members. In general, all the major previously described plant transposon families were represented—class I: Ac-like, En/Spm-like, Mutator, and MITEs; class II: copia-like retrotransposons, gypsy-like retrotransposons, LINEs and SINEs. RESites could be identified from several members from the larger groups (FIG. 1A). However, 179 VIRMIN transposons could not be classified into these groups. Furthermore, there is a high degree of sequence diversity suggesting that most, if not all, of the groups are older components of the genome.

In FIG. 1A, the target sequences are underlined and the TSDs are shaded. GenBank gi numbers and nucleotide position on clones are indicated. The symbol “¶” indicates the target sequences that are inserted into a Basho III element. The symbol “‡” indicates the target sequences that are inserted into a Basho III element. The symbol “*” indicates the target sequences that are inserted into a MITE IX element.

TABLE I
Transposons in 17.2 Mb of the Arabidopsis thaliana genome

| Type | Superfamily | # of groups | # of transposons |
|---|---|---|---|
| Class I | | | |
| | SINEs | 3 | 16 |
| | LINEs | 28 | 51 |
| | copia-like | 27 | 40 |
| | retrotransposons | | |
| | gypsy-like | 23 | 45 |
| | retrotransposons | | |
| | undetermined | 2 | 2 |
| Class II | | | |
| | Ac-like | 7 | 38 |
| | CACTA-like | 1 | 3 |
| | MULEs | 28 | 108 |
| | MITEs | 15 | 105 |
| | Mariner-like | 1 | 56 |
| Class ? | | | |
| | Basho | 7 | 179 |
| Total | | 142 | 623 |

For many large plant genomes, numerous class I elements, namely copia-like retrotransposons, have accumulated within intergenic regions to the extent that they can make up a significant percentage of the total genome. Class I elements mined with the method of the present application were for the most part truncated which is consistent with a previous study examining retrotransposon sequences located in close association with plant genes. The reverse transcriptase domain of copia-like retrotransposons, gypsy-like retrotransposons, and LINEs were commonly annotated in the sequence files, especially in the large Arabidopsis and rice sequenced clones. However, the actual regions corresponding to these elements were often not reported. LINEs and SINEs that predominate mammalian genomes are represented but make up only a small percentage of the total of VIRMIN transposons.

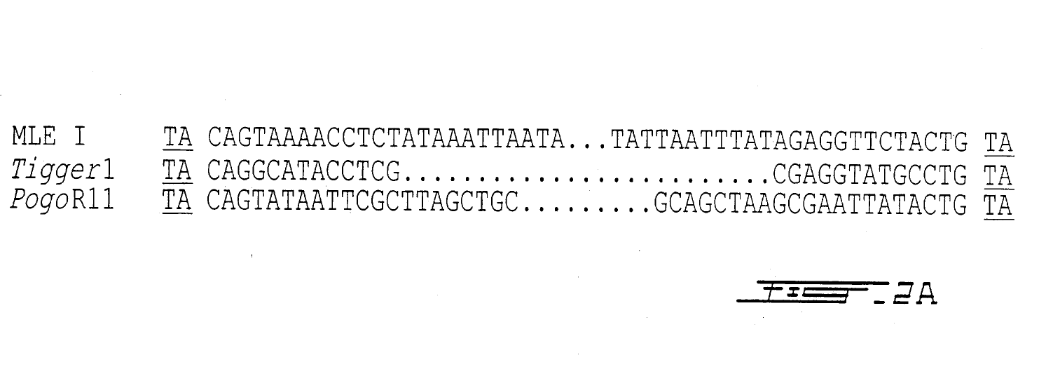

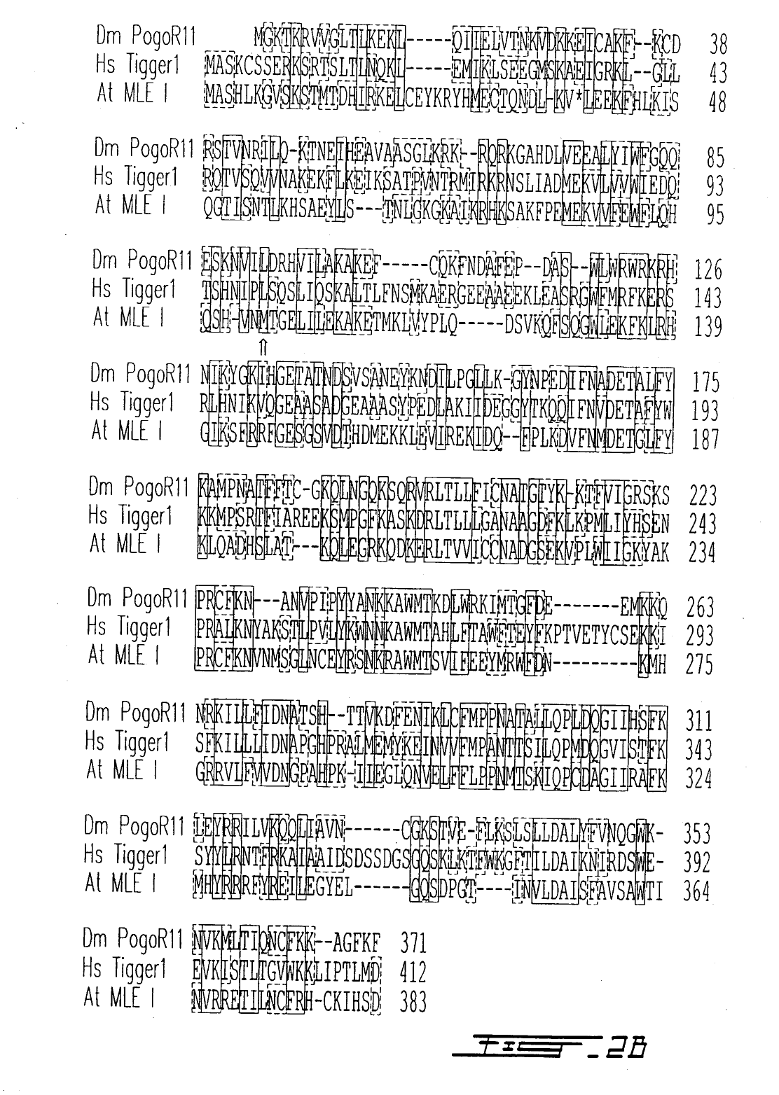

Class II elements are clearly the most prevalent type of transposon found in plants. Ac-like elements are well represented and some members have putative open reading frames (ORFs) coding for an Ac-like transposase. All of the Ac-like elements have terminal inverted repeats (TIRs) similar to other previously described Ac-like elements. Despite reports of En/Spm-like elements in several plant species, only a few elements were mined with the method of the present invention. MITEs are by far the most numerous transposon in plants. Many of the previously reported MITE families are represented. Interestingly, the Tourist family was previously reported as only being found in monocot plants. The study carried out for the present invention indicates that Tourist and Tourist-like families are well represented in Arabidopsis. In addition, one group of mined elements (MLE I) not only shares structural features with the Tc1/Mariner transposon superfamily (FIG. 2A), but also has at least one member located on chromosome 2 that harbors an ORF with up to 46% amino acid sequence similarity with the transposase of Tc1/Mariner-like elements, PogoR11 and Tigger1 (FIG. 2B). In FIG. 2B, similar residues shared between all three sequences are shaded in black while residues conserved between two sequences are shaded in grey. The arrow ( [symbol] ) indicates the predicted start of the Arabidopsis MLE I ORF as annotated in GenBank ( [symbol] ). The first methionine of the Arabidopsis MLE I transposase was inferred from the reading frame and sequence similarity with the human Tigger1 element. The stop (*) was introduced by a single nucleotide substitution (at position 85709 in gi 4262209) from GAG (glutamine) to TAG (stop).

Furthermore, MLE I elements have the conserved terminal bases necessary for the efficient transposition of other Tc1/Mariner-like elements. Some members of the MLE I have been reported to belong to a novel family of MITEs, referred to as Emigrant, based on their small size and target site preference for the dinucleotide TA. However, the MLE I elements clearly have more in common with transposons of the Tc1/Mariner superfamily (FIGS. 2A and 2B) than to elements belonging to the MITE superfamily. The mined MLE I transposase shares no significant sequence similarity with two degenerate Tc1/Mariner-like transposases reported by Lin et al. (Lin, X. et al., Nature 402:761-768, 1999) also on chromosome 2.

Several elements of the class I identified with the method of the present invention were structurally related to the maize Mutator transposon. These elements are referred to as Mutator-like elements or MULEs. MULEs have long TIR sequences ranging from 50 to 300 base pairs, a 9-10 base pairs target site, and some elements contain ORFs with significant amino acid similarity to the maize MuDRA transposase. With the method of the present invention, 32 MULE subfamilies could be identified in Arabidopsis alone. Some Arabidopsis MuDRA-containing MULEs also harbor additional ORFs. Two MULEs harbor partial cellular sequences with high similarity to transcription factor genes. Lastly, two MULE subfamilies do not have TIRs. Despite this, these elements still have a 9 base pair target sequence, as confirmed by the identification of insertion polymorphisms, and some members harbor MuDRA-like ORFs.

Over one-third of the transposons mined with the method of the present invention could not be classified into any of the known plant transposon superfamilies. Some of these were small novel class I element families. Surprisingly, however, many of unclassifiable transposons belong to one novel family. The previously described repetitive sequences referred to as Aie (Arabidopsis insertion element) and AthE1 (Arabidopsis element 1) (Surzycki and Belknap, Journal of Molecular Evolution 48: 684-691, 1998) have nucleic acid sequence similarity to some members of this family. In addition, some of the family members have been annotated as being repetitive (e.g. found on more than one BAC or PAC clone) by the laboratories participating in the Arabidopsis Genome Initiative (AGI)(Lin et al. supra, Mayer et al., Nature 402:769-777, 1999). With the method of the present invention, 179 members of this family which have been named Basho (after the nomadic Japanese poet and father of the haiku form), have been mined. Basho elements in Arabidopsis fall into nine distinct subfamilies based on sequence similarity.

Despite the fact that sequence annotation from AGI and two previous reports suggests that sequence corresponding to some members of the Basho family were repetitive, no evidence was given that these fit the profile of a transposon. In order to establish that Basho was bona fide transposon, several RESites indicating past Basho mobility were identified. In addition, these RESites indicate that target site of insertion for Basho elements is the mononucleotide “T”. The RESites also indicate that Many Basho elements have a short terminal repeat of two or three base pairs. In addition these elements have no sequence similarity to any class 1, 2, or 3 gene suggesting that it is not a mobilized transcript (e.g. SINE or processed pseudogene). They also lack a poly-A rich 3′ end indicative of many mobilized transcripts. Basho elements have a significant potential to form complex RNA or DNA secondary structure.

Surprisingly a group of five Basho-like elements were also mined from maize genomic gene sequences. The maize elements share many of the general structural characteristics of the Arabidopsis Basho elements. However, they share no significant sequence similarity except at the extreme termini. Maize Basho elements appear to also have a past mobile history and a target site preference for the mononucleotide “T” (FIG. 1B). The presence of Basho elements in two divergent plant species, that is in dicotyledonous and monocotyledonous plants, suggests that Basho or Basho-like elements are likely to be present in most plant genomes. The maize and Arabidopsis elements therefore represent a novel superfamily of elements referred to as the Basho superfamily.

In FIG. 1B, RESites found for Basho insertions confirm mononucleotide TSD (shaded). The symbol “†” indicates that the sequences were inserted into a Basho V element.

### General Purposes and Commercial Applications

Various studies have shown transposable elements to be present in virtually every species studied to date. Retrotransposons are present in plant genomes in high copy numbers. The Alu family was estimated to be 5×10⁵ copies per haploid human genome that translates to one Alu element in every 5 kb of DNA. This element alone accounts for 5% of the genome in primates. Ty1/copia group elements can accumulate up to 10⁶ copies per genome in Vicia species, making up to >2% of the genome, although wide variations were seen across species. The BARE-1 retrotransposon has a copy number of 3×10⁴ and makes up to 6.7% of the barley genome. Sequencing of a contiguous 280-kb region flanking the maize Adh1-F gene isolated on a yeast artificial chromosome (YAC) clone revealed 37 classes of nested retrotransposon repeats that accounted for >60% of the clone. As documented in current mining study and in previous reports many genes are associated with members of the MITE superfamily of transposons.

The ubiquity and dispersion throughout the genome of transposable elements suggest that they can be exploited as PCR-based mapping tools. Indeed, Alu-specific primers can be used in search of polymorphisms among different human DNA samples. These investigators clearly demonstrated the feasibility of using these polymorphisms (termed alumorphs) as a genome analysis tool and successfully used these alumorphs to detect the linkage of one alumorph to a human disease (Zietkiewicz et al., Proceedings of the National Academy of Sciences (USA) 89: 8448-8451, 1992). A copia-like retrotransposon, PDR1, was also successfully used to study polymorphisms and, in combination with other specific primers, to diagnose different lines in Pisum (Flavell et al., Plant Journal 16: 643-649, 1998). MITEs have been successfully exploited in a novel technology called inter-MITE Polymorphism (IMP) as mapping and fingerprinting tools in barley.

Mining of novel transposons offers the possibility to develop a method for a high-throughput screen of active endogenous transposons. This method would be universally applicable to any plant species were there is sufficient DNA sequence information available to mine transposons. Importantly, transposon information can be mined from the targeted plant species or from related plant species. Active endogenous transposons would be identified using conditions optimized for maximum mobility—that is under stress conditions. Three stresses in particular have been documented to activate transposons, namely protoplast formation, ultraviolet-B (UV-B; 280-320 nm) radiation, and Agrobacterium infection. Elements chosen for analysis will be based on whether they harbor ORFs encoding mobility-related proteins, are members of groups sharing high sequence similarity, and/or have RESites documenting recent mobility.

These technologies are clearly limited only by the identification of new transposons. The present invention details an efficient method for mining bona fide transposons from nucleic acid sequence databases. VIRtually MINed (VIRMIN) transposons will clearly facilitate the development of new powerful genome analysis tools and in the identification of transposons for gene tagging and gene knockout protocols central to functional genomics. Clearly, the methodology and subsequent database construction and deposition will be of enormous value to the development of downstream biotechnologies.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method for determining a value indicative of a nucleic acid sequence being a transposon, the method comprising the steps of:
a) identifying a location in a nucleic acid database at which a potential transposon to be identified may be found;
b) selecting at least one flanking region sequence of said potential transposon;
c) searching said database for at least one match of said at least one flanking region sequence;
d) comparing a target site nucleic acid sequence and both leading and trailing ones of said flanking region sequences between said potential transposon and said at least one match.
e) determining said value as a result of step d).

2. The method as claimed in claim 1, wherein step a) is completed by querying a nucleic acid database to find repetitive sequences, queries including genomic sequences being selected from the group consisting of non-coding regions, regions annotated with low similarity to genes or with predicted exons, sequences annotated as previously identified transposon, sequences annotated as having an open reading frame as part of a previously identified transposon, sequences annotated as having a putative transposon and sequences annotated as having a repetitive region, said queries being executed with one or more search algorithms and said queries retrieving regions with significant sequence similarity.

3. The method as claimed in claim 2, wherein said search algorithm is Basic Local Alignment Search Tool (BLAST).

4. The method as claimed in any one of claims 2-3, wherein step a) is also completed by screening sequences for structures indicative of transposons, said structures including terminal inverted repeats (TIRs), long terminal direct repeats (LTRs), genes related to mobility and Target site duplications (TSDs), said screening using one or more structure identifier algorithms facilitating structural analysis.

5. The method as claimed in claim 4, wherein said structure identifier algorithms are GAP, REPEAT and STEMLOOP.

6. The method as claimed in any one of claims 1-5, wherein said value indicative of a nucleic acid sequence being a transposon is based on correspondence of insertion sequence to a gap in pairwise alignment coupled to the presence of a target site duplication, said correspondence being determined using sequence similarity criteria.

7. A computer program product comprising code means adapted to perform all steps of any one of claims 1 to 6, embodied on a computer readable medium.

8. A computer program product comprising code means adapted to perform all steps of any one of claims 1 to 6, embodied as an electrical or electro-magnetic signal.

9. A computer data signal embodied in a carrier wave and representing sequences of instructions which, when executed by a processor cause the processor to perform all steps of any one of claims 1 to 6.

10. An apparatus for determining a value indicative of a nucleic acid sequence being a transposon comprising:
means for identifying a location in a nucleic acid database at which a potential transposon to be identified may be found;
means for selecting at least one flanking region sequence of said potential transposon;
means for searching said database for at least one match of said at least one flanking region sequence;
means for comparing a target site nucleic acid sequence and both leading and trailing ones to said flanking region sequences between said potential transposon and said at least one match;
means for determining said value as a function of said comparing.

11. The apparatus as claimed in claim 10, wherein identifying a location in a nucleic acid database is completed by querying a nucleic acid database to find repetitive sequences, queries including genomic sequences being selected from the group consisting of non-coding regions, regions annotated with low similarity to genes or with predicted exons, sequences annotated as previously identified transposon, sequences annotated as having an open reading frame as part of a previously identified transposon, sequences annotated as having a putative transposon and a sequence annotated as having a repetitive region, said queries being executed using one or more search algorithms and said queries retrieving regions with significant sequence similarity.

12. The apparatus as claimed in claim 11, wherein said search algorithm is BLAST.

13. The apparatus as claimed in any one of claims 10-12, wherein identifying a location in a nucleic acid database is also completed by screening sequences for structures indicatives of transposon, said structures including TIRs, LTRs, genes related to mobility and TSDs, said screening using a structure identifier algorithm facilitating structural analysis.

14. The apparatus as claimed in claim 13, wherein said structure identifier algorithms are GAP, REPEAT and STEMLOOP.

15. The apparatus as claimed in any one of claims 10-14, wherein said value indicative of a nucleic acid sequence being a transposon is based on correspondence of the insertion sequence to a gap in pairwise alignment and the presence of a target site duplication, said correspondence being determined using sequence similarity criteria.
